(19) United States
(12) Patent Application Publication
(10) Pub. No.: US 2024/0269094 A1
(43) Pub. Date: Aug. 15, 2024

(54) Treatment of canine dermatitis and skin irritation

(71) Applicant: Edward Tak Wei, Berkeley, CA (US)

(72) Inventor: Edward Tak Wei, Berkeley, CA (US)

(21) Appl. No.: 18/445,117

(22) Filed: Apr. 14, 2023

Related U.S. Application Data
(63) Continuation-in-part of application No. 63/576,281, filed on Jan. 27, 2023.
(63) Continuation-in-part of application No. 63/576,781, filed on Mar. 2, 2023.

(51) Int. Cl. A61K 31/16; A61K 9/00; A61K 36/534; A61P 17/04

(52) U.S. Cl. CPC A61K 31/16; A61K 9/0014; A61K 36/534; A61P 17/04

(57) ABSTRACT

The discovery is of a cooling molecule and its formulation as a topical medication for treatment of skin dysesthesia in hair-coated mammals. Hair-coated mammals such as dogs are seldom treated with topical medications because the hair on the skin and the stratum corneum prevents drug access to receptors in the epidermis. In screening cooling agents for their actions in the oral cavity and throat in humans, a FEMA (Flavor Extract Manufacturers Association) entity, registered for use in chewing gum and hard candy, produced an acute stinging and painful cold, like ice chips in the throat, which was long-lasting. These actions suggested that it might have anti-itch properties. The FEMA entity was formulated as a topical gel or as a liquid, and these formulations relieved skin itch in humans and dogs without causing any skin injury. The duration of the cooling action, ≥3 hr, was sufficient to be therapeutically effective. In dogs, the FEMA entity relieved itchy stimuli from the inflammation of allergies and stopped scratching behavior in subjects with canine dermatitis. Other related FEMA cooling agents used in the oral cavity as comestibles or in toiletries such as WS-3, WS-5, WS-12, and WS-23 were ineffective in the therapeutic relief of canine dysesthesia primarily because of a limited duration of action. Topical formulations for cooling relief of skin dysesthesia in hair-coated mammals represent a new strategy for treatment.

### CROSS REFERENCE TO RELATED APPLICATIONS

This application claims the benefits of a Provisional Application 63/576,281 filed Jan. 23, 2023, and a Provisional Application 63/576,781 filed Mar. 2, 2023.

### BACKGROUND OF THE INVENTION

### Canine Dermatitis—Introduction

The domesticated dog (Canis familiaris) has about 200 breeds recognized by Kennel Clubs. The human-dog bond is firm and eternal because the dog is such a good companion. However, care for the dog's health requires resources. The dog's skin is its largest organ, about 12 to 24% of body weight. In most breeds, hair covers the skin except on the footpads and nose. The density of canine hair is about 15× that of the human scalp, and each hair follicle has a sebaceous gland. Skin disorders most frequently initiate a dog owner's visit to the veterinarian.

The dog's skin is close to the ground, exposed to dirt, hard to keep clean, attacked by insects and microorganisms, and has an immune system that vigorously reacts to allergens. Common disorders in dogs are bites from fleas and mites, infections of the hair follicles by fungi and bacteria, and allergies. In addition, the genetics of the breed influence skin sensitivities. For example, the West Highland Terrier and Labrador Retriever are susceptible to atopic dermatitis. Spaniels, because of droopy ears, are susceptible to infections of the pinna, and Bulldogs are to irritation of the facial skin folds.

Hair-coated animals cannot describe their skin sensations in words. However, they point to the source of skin discomfort and irritation by licking, biting, and scratching. The general term “dysesthesia” encompasses reactions to noxious stimuli such as irritation, burning sensations, itch, and pain. Licking and rubbing may be a form of cleansing the site of dysesthesia. Itch is the sensation that makes us want to scratch and may be particularly annoying to the dog because of its coat. That is, the dog cannot “get to” the site of irritation. Biting an itchy site to cause pain may be preferable to the itch. However, biting and scratching exacerbate skin injury. A vicious “itch-scratch cycle” prevents the skin from healing. If scratching diminishes, the skin heals faster. Cooling gels in humans reduce itch but have not been used in dogs because the dog's hair impedes the access of the coolant to the skin receptors.

### Canine Dermatitis, Sites of Scratching

The canine sites of dysesthesia with thinner skin and less hair are on the belly, armpit (axillary), groin (inguinal), and raw or “hot spots” on the hindlimbs, the back, and at the base of the tail. Areas with thicker skin are the ear canal, neck area, and the skin between the toes (interdigital). Skin sensations at all sites are disrupted by disorders such as atopic dermatitis, atopic-like dermatitis, allergens, fungi, bacteria (folliculitis, pyoderma), and acute and chronic reactions to bites from insects and parasites. The cutaneous inflammatory response to injury is as important as the injury itself. For example, for bites from insects or arachnids, it is not the withdrawal of blood by the flea or the mite's consumption of cellular debris that causes injury. Instead, it is the host's allergic reactions to the invading arthropod's saliva, eggs, larvae, and feces.

### Current Drug Therapy for Canine Dermatitis

Current drug treatments for canine skin disorders are sophisticated, a multi-billion dollar business, and require accurate diagnosis and targeted therapy. The drug management strategy can be specific or general. For example, to treat scabies and other mites, use a topical scabicide such as permethrin, malathion, or lindane. For fleas or ticks, an orally ingested neurotoxin, afoxolaner (e.g., Nexgard), will poison the flea or tick when it drinks the dog's blood. A topical or systemic antibiotic is used for bacterial or fungal infections of hair follicles (e.g., hot spots or pyoderma traumatic) or skin. Prescription drugs with a generalized effect on the immune system are for allergies. For example, an effective immunosuppressant in atopic or atopic-like dermatitis is the specific JANUS kinase inhibitor called oclacitinib (Apoquel). Other immunosuppressants are cyclosporine or anti-inflammatory steroids. Immunosuppressants, however, have a powerful effect on the body, with a variable onset, and long-term use may lead to undesirable side effects. Topical anti-inflammatory steroids for dermatoses in humans have limited efficacy in the dog and have an additional risk of super-infection.

The dog owner is most concerned when the dog repeatedly licks, bites, and scratches the skin disorder site. A dog's scratch with its claws, alternating contraction and relaxation of the limb muscles, arching of the spine, angulating the head, and targeting the site to scratch, is vigorous and will damage tissues. And so will the bite. The skin damage perpetuates the skin disorder in the “itch-scratch cycle.” Interruption of the itch-scratch cycle will be an effective new method of treatment. In humans, l-menthol is sometimes used in anti-itch remedies. However, menthol is irritating and cannot be used for canine skin. Currently, no topical cooling drugs exist to relieve canine skin dysesthesia or interrupt the itch-scratch cycle.

### BRIEF SUMMARY OF THE INVENTION

The applicant has experience studying cooling agents for treating experimental and human itch. For example, the applicant showed that icilin ointment prevented scratching excoriations in hairless rats on a magnesium-deficient diet (Biro et al., 2005). The activities of p-menthane carboxamides and, more recently, the efficacy of 1-dialkylphosphorylalkanes (DAPA) were explored. DIPA-1-7 (Cryosim-1), a DAPA, inhibited the itch of urticaria and scalp itch in human subjects (Kang et al., 2022). The positive results in treating human scalp itch suggested that the technology may apply to mammals with hair coats. The applicant, therefore, initiated studies to find an agent that will penetrate the hair coat of a mammal and reach cooling receptors in the epidermis.

In adapting this knowledge for canine dysesthesia, these objectives were necessary: a) define the molecular target and mechanism for a drug, b) state the target's anatomic location, and c) develop methods for formulating and delivering the drug to the target. For experiments, the investigator selects the class of chemicals and the bioassay. A milestone occurs when a chemical entity is identified that a) has a rapid onset of action, b) can be produced, formulated, and delivered at reasonable costs, c) is free of adverse side effects, d) has a sufficient duration of action, and e) reduce dysesthesia, inhibits scratching, and progression of the itch-scratch cycle. Overall, the treatment should accelerate skin healing.

In one aspect of the present invention, a pharmacological composition for the treatment of a dermatological disorder in a mammal in need of treatment thereof comprises:

A liquid or semi-liquid composition having a therapeutically effective amount of one or more compounds having Formula 1

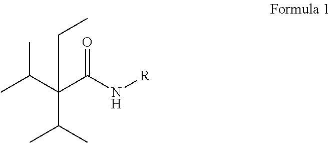

Wherein R is ethyl, n-propyl, isopropyl, cyclopropyl, or acetyl.

The preferred embodiments of Formula 1 are cooling agents that presumably act as an agonist on a TRP channel protein called TRPM8 located on neuronal membranes. More precisely, the preferred embodiment is FEMA 4557, also known as N-ethyl-2,2-diisopropylbutanamide [CAS No. 51115-70-9, N,2-diethyl-3-methyl-2-(1-methylethylbutanamide)]. The agent activates TRPM8 and generates a signal to the brain, interpreted as cooling. This signal suppresses dysesthesia. Surprisingly, there is no direct correlation between the TRPM8 receptor potency of a particular agonist and the duration of cooling. One consequence of using the preferred embodiment is interrupting the itch-scratch cycle. Another surprising finding was that the preferred embodiments were well-tolerated when applied at ultra high concentrations on the skin from 5 to 60% wt/vol in liquid or semi-liquid form. Liquid formulations have the advantages of enabling even distribution of the drug over the skin and drug access to the receptive field in the epidermis, the intended target. In addition, these formulations do not cause avoidance or irritation at the application site that suppresses scratching. Finally, the cooling lasts for at least three hours or longer. This duration is sufficient for therapeutic efficacy. Examples of such formulation and case histories of treatment are presented.

The disorder of interest is canine dermatitis and the skin's reaction to injury, but the invention applies to other hair-coated mammals such as cats and horses. For the dog, the treatment goal is to stop the dog's scratching and to abort the irritant-scratch cycle so the inflamed tissues have time to heal. Other aspects and embodiments of the present invention are further clarified below.

### BRIEF DESCRIPTION OF THE SEVERAL VIEWS OF THE DRAWINGS

FIG. 1. Topical Application of a cooling DIPA1-7 gel on skin of healthy volunteers inhibits itch induced by various pruritogens. n=30 subjects per trial. Results of Prof. Hyeone Kim, Sacred Heart Hospital, Hallynm University, Seoul, Korea. Itch intensity was self-rated on a scale of 0 to 10 for 10 min (maximum score=100). DIPA1-7 (Cryosim-1) gel (Intrinsic IB, Dong Wha Pharmaceuticals, Seoul, Korea) significantly inhibited itching induced by all five test pruritogens. Further details will be described in a scientific paper.

FIG. 2. Cooling intensity of agents tested in the oral cavity at 100 ppm in 5% sucrose. Cooling intensity was self-rated on a scale of 0 to 9 units. Data from FIG. 4 of Sonya Johnson et al., 2018. The rank order of potency of WS-5>WS-3˜l-menthol>WS-12>WS23 agrees with compilations published by Leffingwell (2014) and by Erman (year).

FIG. 3. Median Effective Dose (EC₅₀) of various cooling agents in TRPM8 in vitro assay. The consensus is that icilin and WS-12 are the more potent agonists, followed by WS-5>WS-3˜l-menthol. The acyclic amides WS-23 and FEMA 4557 are significantly less active than l-menthol. The applicant provided the test substances used in the publications by Mckemy et al., Bodding et al., and Sherkheli et al. Further experiments by the applicant confirmed the weak activity of FEMA 4557 on TRPM8 (10% the activity of l-menthol) and the absence of activity on TRPA1.

FIG. 4 Bioassay of FEMA 4557 (labeled as BUDA on the graph) in TRPM8 and TRPA1. Data from the laboratory of Prof. Seok-Yong Lee at Duke University. The EC₅₀ of FEMA 4557 was estimated at 0.41 mM or about 1/30× that of l-menthol. FEMA 4557 was not active in cells transfected with TRPA1, although a positive TRPA1 control AITC (allyl isothiocyanate) was active on TRPA1.

FIG. 5. The philtrum bioassay. Cooling agents, prepared as a or in solution are applied to the philtrum skin. Cooling intensity is rated on a scale of 0 to 3, with 0=no changes in sensation, 1=a slight feeling of coolness, cold or tingling, 2=a clearcut feeling of coolness of cold, and 3=a robust feeling of coolness of cold. The duration of cooling was recorded until two zeroes were measured 10 min apart.

FIG. 6. Cooling agents, prepared as an 8% wt./vol. gel, was tested topically on the philtrum skin assay of volunteers. The duration of cooling was recorded (min on y-axis). The startling activity of FEMA 4557, cooling for ˜3 hr, was unexpected and surprising. FEMA 3784 is also known as Cool 10 or menthoxypropanediol.

FIG. 7. Dose-response for FEMA 4557 on Philtrum Assay.

FIG. 8. A pen-like delivery system for administering the active ingredient to lesion sites.

FIG. 9. Examples of canine irritated sites for topical treatment: belly and groin, and interdigital skin folds.

### DETAILED DESCRIPTION OF THIS INVENTION

### Cooling Agents: Metamorphosis of Menthol to FEMA 4557

In searching for a molecule that suppresses skin irritation after topical application, it is essential to identify the best chemical ingredient using an appropriate bioassay.

Drug discovery usually begins with herbal remedies, but when Friedrich Serturner crystallized morphine from opium resin in 1804, the rationale for using botanical mixtures faded and modern pharmacology emerged. Now, we think of the active ingredient as a molecule of defined chemistry, not a plant concoction of unknowns. Once isolated and characterized, the active ingredient becomes a seed for a slow metamorphosis, a glacial evolution, into an improved or better entity. Witness salicylic acid in willow bark become aspirin, then ibuprofen. After Serturner crystallized morphine from opium resin, the metamorphosis spawned codeine, heroin, oxycodone, methadone, and fentanyl.

The efforts to find better analogs from a prototype always entail extensive synthesis and bioassay, a common yardstick of synthesis being 1000+ analogs. Paul Ehrlich, the father of modern pharmacology, called his anti-syphilitic salvarsan 606 because it was the 606th analog! Ehrlich then made an improved neosalvarsan after 900+ analogs and assays. Another example of synthetic efforts at “finding the right needle in a haystack” was Gerhard Domagk, who tested thousands of sulfonamides before finding Prontosil rubrum (which then progressed to sulfanilamide). Adams and Nicholson also screened thousands of compounds before finding ibuprofen. The prototype before ibuprofen was aspirin, which in turn is an analog of salicylic acid, first discovered as an analgesic in willow bark by Reverend Edward Stone in 1763. Drug discovery requires luck and effort. The discovery statistics are such that one would be fortunate to find ten candidates (1%) after making a thousand and refining one out of 10 (0.1%) to a lead candidate. And that lead candidate will likely fail in four out of five clinical trials!

TABLE 1
Representative TRPM8 agonists of major classes of cooling agents.

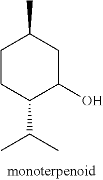

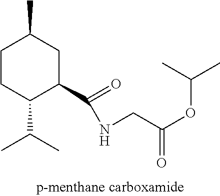 [SMILES: CC(C)OC(=O)CNC(=O)[C@@H]1C[C@H](C)CC[C@H]1C(C)C]

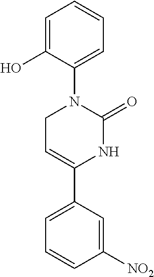 [SMILES: O=C1NC(c2cccc([N+](=O)[O-])c2)=CCN1c1ccccc1O]

icilin

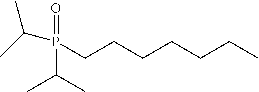

1-dialkylphosphorylalkane
TRPM8 agonists. Clockwise: I-Menthol, Ax-8, icilin, DIPA-1-7

The four major classes of TRPM8 agonist are monoterpenoids (represented by menthol), p-menthane carboxamides (represented by WS-12 and Ax-8), icilin, and 1-dialkylphosphorylalkane (DIPA-1-7, cryosims) (Table 1). With the exception of DIPA-1-7 (Cryosim-1), none will work for the itch of canine dermatitis. Menthol is a skin irritant. Icilin will not dissolve and can't be delivered. The large class of p-menthanecarboxamides are not easily soluble and have limited penetration of skin. DIPA-1-7 will work on itch in humans, but it may be too expensive for use as a drug on dog skin.

For menthol, Wilkinson Sword (WS) scientists tried to improve menthol 50+ years ago and made 1200+ analogs. Watson et al. (1978) summarized the results in a classical paper. The WS effort ended in 1980. Leffingwell and Rowsell (2014) re-visited and updated this subject; by then, thirty-two cooling agents were in the FEMA (Flavor Extract Manufacturers Association, founded in 1909) database. FEMA-designated WS agents are GRAS (generally recognized as safe) and intended for use in the oral cavity. These agents are preferred for screening skin because FEMA has collected safety data. The WS agents assigned a FEMA number are periodically reviewed and information posted online. Thus, the preference is for the FEMA label over the WS label.

The first intended use of WS analogs was for after-shave lotions (skin applications), but this shifted to cooling of the oral cavity. Today, metric tons per year of WS-3, WS-5, WS-12, and WS-23, together with menthol, are used as cooling ingredients in toothpaste (˜45%), chewing gum and confectionery (˜45%), and skin cosmetics and toiletries (˜10%) (Table 2). N-Alkoxyalkyl-substituted WS-23 analogs were described by Sun (2006), but not developed. Recently, WS-5, WS-23, and FEMA 4557 (in China) entered the market as cooling substances in liquids for vaporization in e-cigarettes. However, no WS compounds ever became a therapeutic agent for canine skin irritation management. FEMA 4549 and FEMA 3784 (menthoxypropanediol) were tested in clinical itch trials in humans but did not enter development. The “WS” designations for WS-3, WS-5, WS-12, and WS-23 are familiar from past usage. But the FEMA number is the preferred term for FEMA 4557 because of the online FEMA library.

In this discovery, sensory activity in the oral cavity was the first screen. The goal was to identify candidates producing unusual sensations. Test agents, prepared as 15 mg of white crystals, were placed on the dorsal surface of the tongue. The chemical structures and identifying characteristics are in Table 1. Surprisingly, FEMA 4557 (N-ethyl-2,2-diisopropylbutanamide, WS-27) stood out as a potent agent producing stinging, freezing, and painful cold on the throat. Related cooling agents were not freezing cold, except for WS-23. In later studies, WS-23 was found to be too short-lived for treatment of skin irritation.

### Dermatological Applications of Cooling Agents and FEMA 4557

Extensive hair on the skin is called the “coat” of the organism. The term “hair-coated” is not used for humans. The coat of the domestic dog refers to the hair that covers its body. The coat usually comprises two layers: a topcoat of stiff guard hairs that help repel water and shield from dirt and an undercoat of soft down hair to provide insulation. Although “fur and hair” are frequently used interchangeably, a dog with both coats generally has a “fur coat,” and a single coat is a “hair coat.” For example, a West Highland Terrier or a bulldog has double coats, but the Bichon Frise, which looks like it has much hair, only has a hair coat. Dogs without an undercoat shed less and are considered less allergenic. Cats and horses have a two-layered coat; however, a horse's coat is not called “fur.”

Hair-coated animals are susceptible to skin irritation and dermatoses because keeping the hair and skin clean is difficult. Scratching is typical in cats and dogs. Horses with irritated skin cannot scratch but groom each other and rub their skin against surfaces. By observing human descriptions and behavior, one can infer that hair-coated mammals experience itch and hence are candidates for treatment with FEMA 4557. Treatable mammals include, for example, a rodent (e.g., a guinea pig, a hamster), canine (e.g., a dog), feline (e.g., a cat), equine (e.g., a horse), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human. Effective topical treatment of canine, feline, and equine itching is an unmet need.

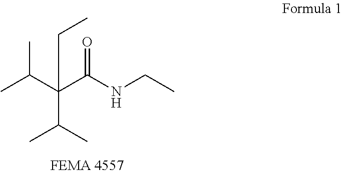

In the oral cavity screen of FEMA agents with cooling properties, FEMA 4557 was identified as an entity of high priority. It was then formulated into a topical gel or solution and applied to the skin surface. The FEMA 4557 formulations effectively treated the itch in canine subjects with insect bites, dermatitis, and allergies. In the skin screening assay, related FEMA agents were not effective. The effective concentration for FEMA 4557's topical activity on the skin is ≥5%, which is higher than those used commercially in the oral cavity. However, skin tissue irritation or inflammation was not observed or detected. The use of concentrations of >5% wt/vol, up to 50% wt/vol, is a surprising aspect of this discovery. The average use levels (ppm)/average maximum use levels (ppm) for FEMA 4557, at which the FEMA Expert Panel based its judgments that the substances are generally recognized as safe (GRAS), is 3000/6000 ppm (or 0.3 to 0.6%) for FEMA 4557 in chewing gum, and 1000/1500 for confectionery frostings and hard candy. The skin tests confirmed and revealed the therapeutic value of FEMA 4557 as a topical medicament.

An example of a topical gel formulation of FEMA 4557 consisted of 8% wt/vol of FEMA 4557, 2% ethanol plus 1,2-propanediol as solvents, and 0.5% carbomer as a gelling agent, and 1% l-methanol as a dispersing agent or 0.2% DIPA-1-7 as an initiator. This gel, applied topically, is effective for 3+ hours with an onset of less than 3 min. A plastic reservoir bottle with a small rounded tip or an airless pump is ideal for dispensing the gel for canine skin. In summary, we have identified a known GRAS flavoring agent as an effective pharmaceutical ingredient for itch, and this is the basis of this discovery.

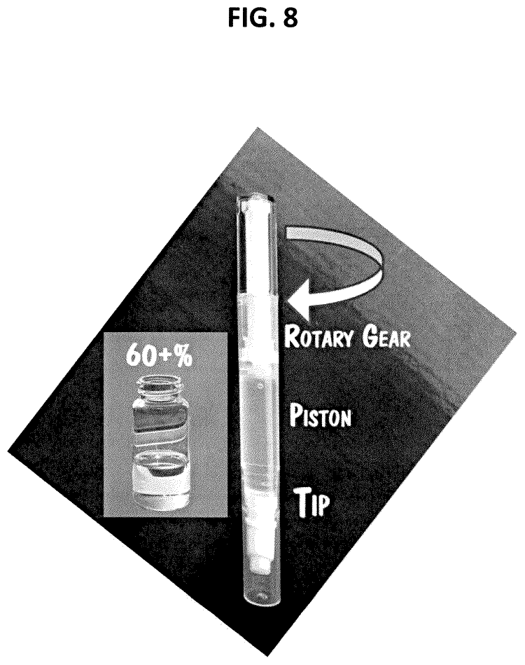

An example of a topical liquid formulation of FEMA 4557 consisted of a 32% wt/vol of FEMA 4557 in 1,2-propanediol, with 4% ethanol and 4% l-menthol. Menthol in this formulation accelerates the onset of cooling and anti-itch effect, thus providing instant gratification to the user. In addition, this liquid formulation is easy to apply with a pen equipped with rotary gear (FIG. 8). The solution is effective for scratching caused by bites of ectoparasites and rashes caused by yeast infection on the paws, hindquarters, and at the base of the tail.

### Chemistry of Cooling Agents and Terminology

The prototype cooling agent is menthol, found in plant species of the Mentha family. Today, the active ingredient used is the l-menthol enantiomer. The applicant described the properties of new cooling agents icilin and 1-dialkylphosphorylalkanes, represented by 1-diisopropylphosphorylheptane (synonyms: Cryosim-1, DIPA-1-7: CAS No 1487170-15-9).

TABLE 2
Several classes of chemical cooling agents.
p-Menthanecarboxamide

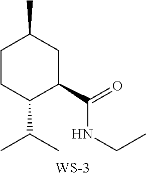

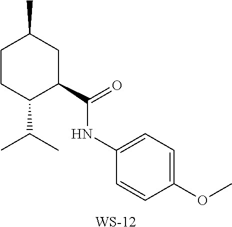 [SMILES: COc1ccc(NC(=O)[C@@H]2C[C@H](C)CC[C@H]2C(C)C)cc1 ; "WS-12"]

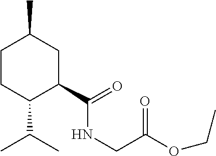 [SMILES: CCOC(=O)CNC(=O)[C@@H]1C[C@H](C)CC[C@H]1C(C)C]

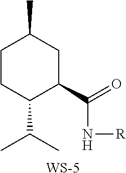

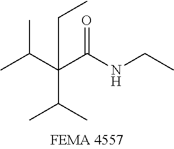

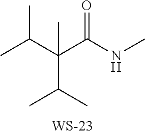

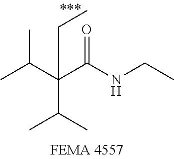

Clockwise: p-menthane carboxamides: where R = ethyl, WS-3; WS-12 where R = p-methoxyphenyl, and WS-5 where R = glycineethyl ester
Acyclic amides, the *** bond FEMA 4557 may act as a “hook” on receptor

The WS compounds most widely used in commerce are the p-menthanecarboxamides (Table 2). Note the p-menthane moiety contains a compact cyclohexane entity. Cyclohexane makes these compounds hydrophobic and difficult to dissolve in aqueous solvents. WS scientists also discovered “acyclic amides” that exerted cooling actions. For the acyclic amide, WS-23 and WS-24 were commercialized. These compounds are “acyclic” because they do not contain the cyclohexane ring. Acyclic amides are generally less potent and short-acting.

Here are comments on grades assigned to specific classes of cooling agents (Table 3). The lower potency of FEMA 4557 does not influence costs because it can be synthesized at low costs. Icilin cannot be formulated for skin delivery: it does not dissolve in any standard dermatological solvent. The p-menthane carboxamides cover a wide range of compounds, but they are difficult for skin formulation to achieve a long duration of action. Menthol is short-acting and irritating, especially when it is near the ocular surface. The 1-dialkylphosphorylalkane known as DIPA1-7 (Cryosim-1, 1-dialkylphosphorylheptane) is an excellent skin agent, but costs are high for dermatological applications.

TABLE 3
Comparison of different classes of cooling
agents for applications on the canine skin.

| | | | | | 1-dialkyl- |
|---|---|---|---|---|---|
| | FEMA | | p-Menthane | l-menthol | phosphor- |
| Properties | 4557 | Icilin | Carboxamides | terpenoids | ylalkanes |
| Ease of | A+ | F | B | B | A+ |
| Formulation | | | | | |
| Ease of | A+ | C | B | B | A+ |
| Delivery | | | | | |
| Rapid Onset | B | B | B to C | A | A+ |
| Potency | C | A | B to C | B | A |
| Sufficient | A+ | A | C | F | B |
| Duration | | | | | |
| Absence of | A | A | A | F | B |
| Irritancy | | | | | |
| Safety in Use | A | A | A | F | B |
| Reasonable | A+ | C | B to C | A | C |
| Costs | | | | | |

A = excellent,
B = average,
C = poor,
F = does not work

Structure Activity: WS-23 and FEMA 4557 have Diisopropyl “Legs.”

The cooling monoterpenoids (menthol group), DAPA compounds, icilin analogs, p-menthanecarboxamides, and acyclic amides can generate a huge number of analogs. In practice, it is impossible to test each analog. But which will work in the clinic? After making a thousand compounds, the first step is to reduce the selection to ten candidates, or 1%. One or two of these ten candidates might have a chance of success in the clinic.

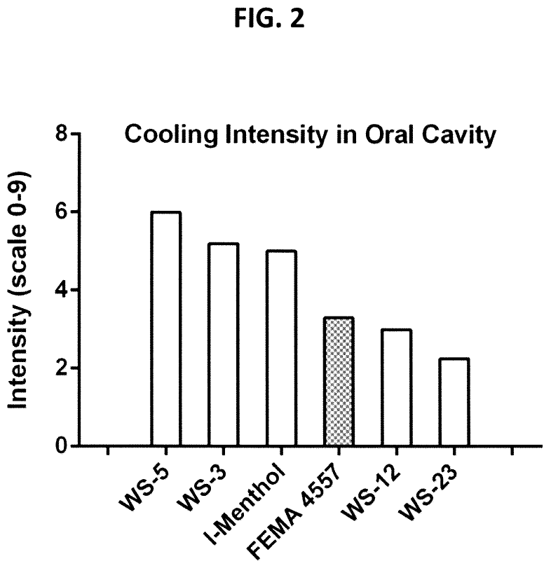

Most data on structure-activity relationships of p-menthane carboxamides is for cooling intensity in the oral cavity (FIG. 2, see Erman et al.; and Johnson et al.). Wei U.S. Pat. No. 6,599,524, 2014, identified some lead candidates for skin in the p-menthane carboxamide series, but these did not enter development. Comparisons of the cooling activities in the oral cavity and skin are in Table 3 and FIG. 2. The cooling intensity does not correlate to the duration of action.

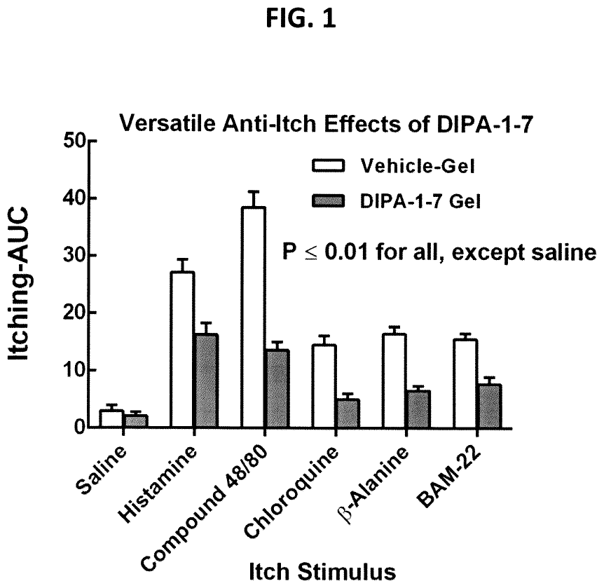

In the DAPA series, only two analogs, DIPA-1-7 and DIPA-1-8, are prime candidates for becoming a drug for human dermatological applications. By selecting these analogs, there was a remarkable success. In the clinic, DIPA-1-7 inhibits itching in patients with urticaria (Jung et al., 2022). DIPA-1-7 also inhibits scalp itch, which is helpful for seborrheic dermatitis and psoriasis (Kang et al., 2022). DIPA-1-7 is approved for such uses in the Republic of Korea. More recent studies show DIPA1-7 as a versatile agent to stop itching (FIG. 1). Pretreatment with DIPA-1-7 prevents itching elicited by five pruritogens that act by different mechanisms of action. DIPA-1-7 is ideal for short paroxysmal itch episodes, such as atopic dermatitis and urticaria. However, for long-term use, DIPA-1-7 has limitations. It is too cold and can be irritating on broken skin surfaces. For treating hair-coated mammals, an ideal alternative to DIPA 1-7 should have a gentler, soothing, and long-acting effect on the skin. The limitations of DIPA-1-7 led the applicant to examine the p-menthane carboxamides and the acyclic amides series and the discovery of the preferred embodiment.

TABLE 4
Isopropyl legs in the structure activity analysis of cooling agents with
the properties of freezing, painful cold.

| | | | | Effects of 15 |
|---|---|---|---|---|
| FEMA | | | | mg on |
| No. | CAS No. | Synonyms | Structure | tongue |
| 2665 | 2216-51-5 | I-Menthol | 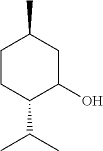 | |
| — | 1487170- 15-9 | DIPA-1-7 (Cryosim-1) | 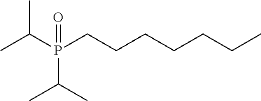 | stinging, painful cold |
| 4557 | 51115- 70-9 | WS-27, N-ethyl-2,2- disiopropyl butanamide, N,2-diethyl-3-methyl-2-(1- methylethyl)-butanamide | 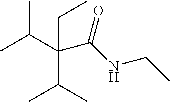 | stinging, painful cold |
| 3804 | 51115- 67-4 | WS-23 | 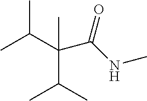 | |
| — | — | 3-methyl-N,2-bis(1- methylethyl)butanamide | 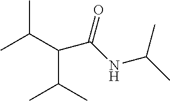 | |

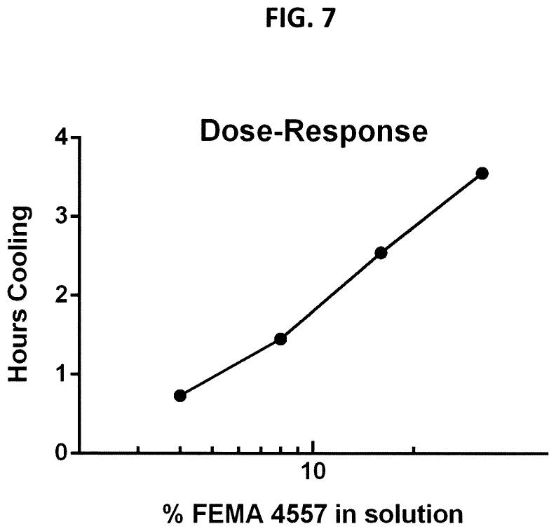

In DIPA-1-7, the symmetrical diisopropyl legs are essential for receptor occupancy and activation (Table 4). Activity goes down when sec-butyl or iso-butyl replaces one isopropyl. WS-23 and FEMA 4557 have symmetrical diisopropyl groups. In this discovery, we find that FEMA 4557 has an unexpected and surprisingly long duration of action. The extra methyl group on FEMA 4557, marked as (***) in Table 2, and which is not on WS-23 or WS-24, may act as a “hook” or “coat-hanger” to trap the molecule at the receptor pocket and prolong occupancy and duration of action (Table 1A). A bonus effect of FEMA 4557 is solubility in biocompatible solvents such as 1,2-propanediol, which is thus easy to formulate and produces comfortable cooling without irritation. Finally, the dose-response relationship for FEMA 4557 (FIG. 7) shows that concentrations ≥5% are ideal for producing cooling for ≥1 hr. This cooling occurs without any adverse effects. Even solutions of ≥32% are well-tolerated on the skin.

The exact binding mechanisms of TRPM8 to receptor sites have now been thoroughly explored in recent publications. Xu et al. (2020) have conducted a thorough analysis of the binding of l-menthol to the mouse TRPM8 receptor. They noted the robust coupling energy between the isopropyl group on the cyclohexane ring and the channel based on the Kd values. For the critical binding amino acid residues, they stated that the strongest coupling energy values for the “isopropyl leg” were at L843 (leucine) and 1846 (isoleucine). In contrast, the V775 (valine) residue outside the binding pocket exhibited smaller coupling energy with the isopropyl legs. Y745 (tyrosine) and F839 (phenylalanine) were within the binding pocket, but apart from the isopropyl legs, they exhibited smaller coupling energy than 1846 and L843. These results suggested that the isopropyl legs point downward as predicted in the docking experiments, where they stand on residues L843 and 1846 to facilitate channel activation.

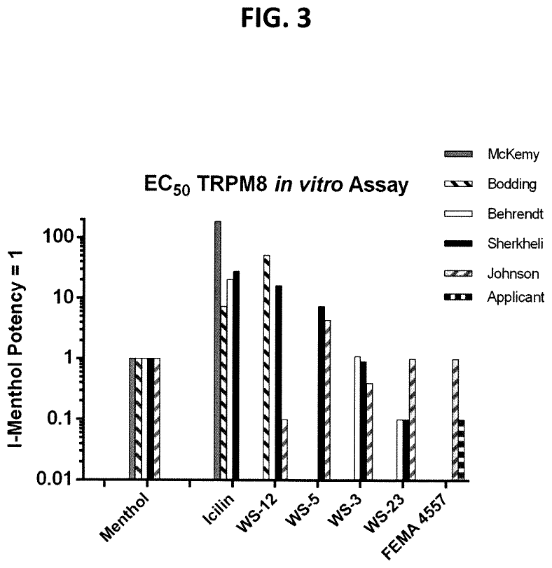

The activity of FEMA 4557 relative to the other compounds tested are shown in Table 2 and FIG. 3. Wei and Co-workers have found that 1-diisopropylalkyl-phosphorylalkanes are potent stimulators of the TRPM8 receptor and inhibit itch. DIPA-1-7 (Cryosim-1), was exceptionally active and stopped the itch of urticarial rashes and scalp itch in human subjects (see Jung and Kang in Refs). DIPA-1-7, at less than 2 mg on the tongue, produces a frigid, stinging, icy, and painful cold, like FEMA 4557. FEMA 4557 and FEMA 3804 are similar in structure (Table 2). Both have prominent isopropyl legs in close juxtaposition to the hydrogen bonding oxygen atom. FEMA 4557 produced potent stinging cold, but not FEMA 3804. The N-ethyl group or the extra methyl group in C-2 of the FEMA 4557 butanamide may fit better into the receptor pocket. Increasing the N-alkyl substituent to n-propyl or iPr may further alter the duration of action. It is of interest to note that out of the 32+ FEMA approved cooling agents, only FEMA 4557 has the property of long duration of action and this may, in part, depend on the presence of the diisopropyl legs and the “coathanger.”

Although the “coathanger” idea provides an explanation for the prolonged activity of FEMA 4557 on the skin, there are alternative possibilities. The high concentrations of FEMA 4557 may also form deposits of crystalline FEMA 4557 after the 1,2-propanediol is absorbed. Such crystals will serve as reservoir for the active to be eluted onto the receptive field. Although no deposits were observed when solutions were applied to cheekbone skin or to the philtrum of the humans, some deposits were noted inside the pinna and ear canal of dogs receiving the 32% solution. The dense hair matrix of the dog skin will be an ideal reservoir for deposit of an anti-itch agent.

### Bioassay for Anti-Itch Effects: Molecular Target and Mechanism

In 2002, groups led by David Julius and Ardem Patapoutian (Nobel Laureates of 2021) showed that menthol and icilin, both purported cooling agents, acted on a neuronal membrane protein subsequently called the TRPM8 receptor. A receptor assay is a standard tool and can be done quickly. For example, in a 384-plate assay, you can easily test 10 compounds and get dose-response data. For a chemical agonist, an in vitro TRPM8 assay generates an EC₅₀ (median effective concentration). But the quantitative correlation between the EC₅₀ on TRPM8 and cooling sensation is not always clear. That is, the language of receptor identification and potency was “roughly” predictive, but there was considerable uncertainty for each agonist. The EC₅₀ values have scientific panache, but do not give information on the duration of action or the quality of the sensation. Table 5 summarizes results on TRPM8 EC₅₀ (see FIG. 3). The specific references for each value are: Ref. 1. Behrendt et al., Ref. 2. Bodding et al., 2007; Ref. 3. Johnson et al., 2018; Ref. 4. Leffingwell et al., 2014; Ref. 5. McKemy et al., 2002; Ref. 6. Sherkheli et al., 2010. The full citations are listed in the References. Leffingwell. Ref. 4 gives the FEMA numbers for each compound.

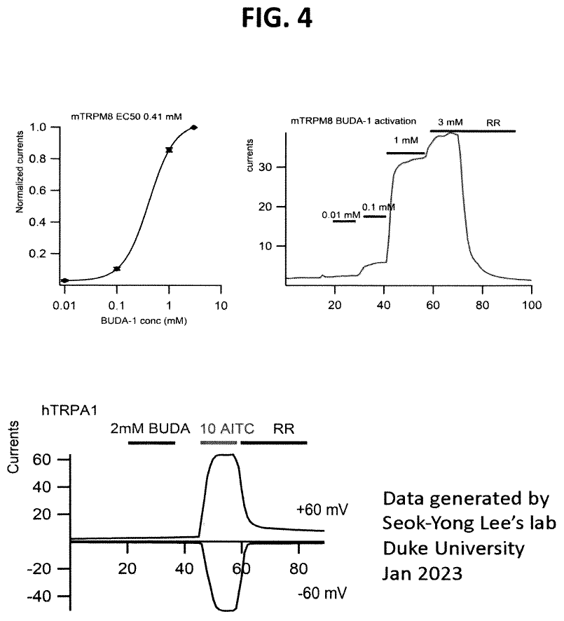

In the context of this application, the TRPM8 EC₅₀ potency number is a misleading dud. If the EC₅₀ value is used as a guide, FEMA 4557 will be an early reject, based on the data of Table 5, because the acyclic amides WS-23 and FEMA 4557 are 0.1 to 1.0 the activity of menthol. For example, Behrendt et al, find that WS-23 has 4.1/44=0.09 the potency of menthol, and values of 196/1500=0.13, and 2.2/2.2=1 were found by Sherkheli and Johnson, respectively. In tests by Prof Seok-Jeong Lee, values of <0.1 potency were obtained (FIG. 4). By contrast, when one examines the cooling actions the order of potency is dramatically reversed. FEMA 4557's cooling duration compared to menthol is >180/25=>7 fold. The TRPM8 EC₅₀ value is not predictive of cooling duration. The philtrum bioassay is the correct measure.

TABLE 5
EC50 of Cooling Agents Tested on TRPM8, See FIG. 3 for graph

| | EC50 | | EC50 | | EC50 | | EC50 | | EC50 |
|---|---|---|---|---|---|---|---|---|---|
| Ref. 1 | μM | Ref. 2 | μM | Ref. 3 | μM | Ref. 4 | μM | Ref. 5 | μM |
| icilin | 0.36 | Icilin | 0.2 | WS-12 | 0.2 | Icilin | 7 | WS-5 | 0.5 |
| menthol | 67 | WS-3 | 3.7 | CPS-113 | 1.2 | WS-12 | 12 | FEMA-4557 | 1.6 |
| | | menthol | 4.1 | Icilin | 1.4 | WS-5 | 26 | menthol | 2.2 |
| | | Cool 10 | 6.0 | CPS-369 | 3.6 | CPS-369 | 65 | WS-23 | 2.2 |
| | | WS-23 | 44 | WS-148 | 4.1 | menthol | 196 | WS-3 | 5.9 |
| | | | | WS-30 | 5.6 | WS-3 | 210 | WS-12 | 22.1 |
| | | | | menthol | 10.4 | WS-23 | 1500 | WS-24 | 34.5 |

FIG. 3. Median Effective Dose (EC₅₀) of various cooling agents in TRPM8 in vitro assay. The consensus is that icilin and WS-12 are the more potent agonists, followed by WS-5>WS-3˜l-menthol. The acyclic amides WS-23 and FEMA 4557 are significantly less active than l-menthol. The applicant provided the test substances used in the publications by Mckemy et al., Bodding et al., and Sherkheli et al. Further experiments by the applicant confirmed the weak activity of FEMA 4557 on TRPM8 (10% the activity of l-menthol) and the absence of activity on TRPA1. The Ref. No. in Table 5 are to: 1=McKemy et al., 2002; 2=Behrendt et al., 2004; 3=Bodding et al., 2007; 4=Sherkheli et al., 2010; and 5=Johnson et al., 2018.

The results of Johnson et al. 2018 (Ref. 5) have the greatest discrepancy from the data of others. Closer inspection and analysis of Johnson's data show the numbers do not fit the Hill Equation criteria for an EC₅₀ measurement. In a Sigmoid Curve, it is essential to have data points above and below the median value, but Johnson's data do not have it. Overall, one can conclude that the acyclic amide coolants, WS-23 and FEMA 4557 have potencies substantially lower (e.g., ⅕ to 1/10) than that of menthol. Thus, the surprising potency of FEMA 4557 against itch is unexpected. The data in FIG. 4 on FEMA 4557 is from Prof Lee's laboratory at Duke University. Prof Lee is the world's leading expert on TRPM8.

FIG. 4 Bioassay of FEMA 4557 (labeled as BUDA) in TRPM8 and TRPA1. Data of Prof. Seok-Yong Lee at Duke University. EC₅₀ of FEMA 4557 of 0.41 mM about 1/30× that of l-menthol. FEMA 4557 not active in cells transfected with TRPA1. A positive TRPA1 control AITC (allyl isothiocyanate) was active on TRPA1.

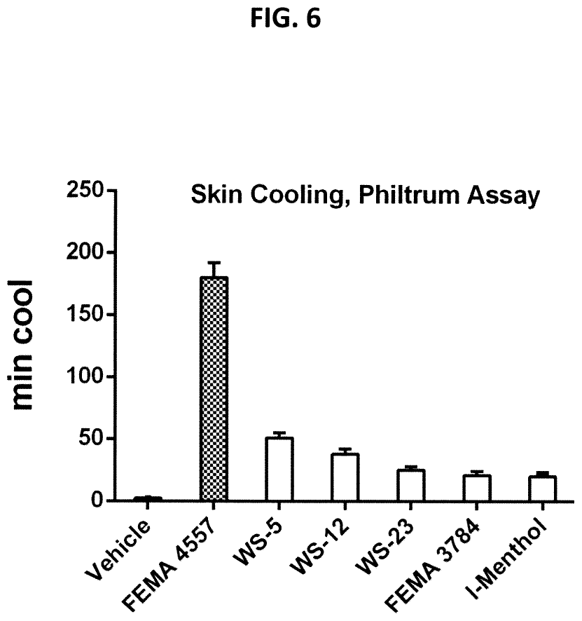

TABLE 6
Cooling in oral cavity and skin. See FIG. 2 and FIG. 6.

| Cooling | Oral Cavity Scaled | Duration- | Hours |
|---|---|---|---|
| Intensity | Johnson, 2018 | Skin | Wei |
| WS-5 | 6 | FEMA 4557 | 3+ |
| WS-3 | 5.2 | WS-5 | 0.5 |
| l-menthol | 5 | WS-12 | 0.4 |
| FEMA4557 | 3.3 | l-menthol | 0.3 |
| WS-12 | 3 | WS-3 | 0.3 |
| WS-23 | 2.25 | | |

Cooling intensity of agents tested in the oral cavity at 100 ppm in 5% sucrose. Cooling intensity was self-rated on a scale of 0 to 9 units (Table 7). Data from FIG. 4 of Johnson et al., 2018. The rank order of potency of WS-5>WS-3˜l-menthol>WS-12>WS23 agrees with compilations published by Leffingwell (2014) and by Erman (year).

The sensory neurons of the skin, studied in isolated dorsal root ganglia neurons, have TRPM8 as the primary protein for detecting cold. However, in about 20% of such labeled neurons, the TRPM8 co-exists with TRPV1 in the same neuron. Co-activation of such TRPM8/TRPV1 neurons may give the sensation of icy, stinging, painful cold. It is essential to recognize that the painful cold help stops itching, not just coolness or cold. TRPA1 is another TRP channel present in sensory neurons and co-exist with TRPM8 and TRPV1. It has been reported that FEMA 4557 activates TRPA1 (Johnson et al. 2018) and is active on the TRPM8. But we were not able to repeat these results. The use FEMA 4557 for the treatment of itch may be based on both pain and cold.

### Screening Tool: Philtrum Bioassay for Finding Topical Active Ingredient

### John Gaddum, Concept of Bioassay and EC50 (Feldberg, 1967).

The rationale of using cooling to gate the perception of noxious stimuli is described in (Kang 2022, FIG. 1). In searching for a molecule that will suppress scratching after topical application, it is essential to have the right bioassays. The importance of bioassay was emphasized by John Henry Gaddum, FRS. Gaddum, a physiologist, became a pharmacologist when he trained with Trevan at Wellcome Research Labs. Trevan standardized the mathematics of the bioassay of digitalis leaf which eventually led to the identification of digoxin, an essential drug for the patient with heart failure. These scientists defined the concepts of the dose-response curve and median effective doses. Gaddum was fond of pithy sayings. Some memorable quotes are:
A branch of science comes of age when it becomes quantitative.
On research. “Enthusiasm is not enough.”
“The pharmacologist has been a ‘jack of all trades’, borrowing from physiology, biochemistry, pathology, microbiology, and statistics—but he has developed one technique of his own and that is the technique of bioassay.”

Trevan and Gaddum recognized that a normal dose-response curve on linear axes was a rectangular hyperbola. The hyperbola became a sigmoid (S)-shaped curve when the x-axis was logarithmic. Further transformation of the y-axis to probit units gave a straight line and facilitated readout of the median effective dose (EC₅₀). This type of pharmacologic analysis is now computerized and routine for in vivo and in vitro assays, such as receptor activation potencies.

Threshold for Cooling on the Surface of the Tongue. Wilkinson Sword in their program for finding better menthol analogs, tested substances by impregnating them on filter paper and applying the filter paper to the tongue of volunteers. The threshold for cooling was first rated as “+++” signs to signify potency and sometimes quantified as “μg” on the tongue. The data were “semi-quantitative” and there is overlap in the potency of many analogs
Intensity of Cooling in Oral Cavity. As WS compounds became widely used in the 1990s in toothpaste, chewing gum, mouthwash, and confectionery, there was a need to re-design the WS bioassay for the oral cavity. The filter paper on the tongue method was replaced by the “hold and spit” method. In this assay, a test substance is suspended or dissolved in a liquid, and the liquid held for 30 see in the mouth and then expectorated. Sometimes, sucrose is added to the test liquid. The sensory effects are recorded by the subject and “cooling intensity” is rated on a scale of 0 to 9. No attempt was made to relate oral cavity cooling to effects on skin. The general consensus was that WS-5 gave the most intense cool, followed by WS-3 and l-menthol, and then WS-12. WS-23 and WS-24 were considered less potent.

TABLE 7
Cooling agents screened for freezing cold activity in the oral cavity. The FEMA
compounds have been evaluated by an Expert Panel and are generally recognized as
safe (GRAS) for its intended use.

| FEMA | CAS | | | Effects of 15 mg |
|---|---|---|---|---|
| No. | No. | Synonyms | Structure | on tongue |
| 4557 | 51115- 70-9 | FEMA 4557 WS-27, N- ethyl-2,2- disiopropyl butanamide | 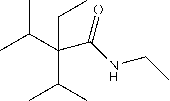 | stinging, painful freezing cold in throat, >35 min |
| 3804 | 51115- 67-4 | FEMA 3804 WS-23 | 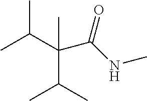 | painful cold in throat, ~18 min |
| 3455 | 39711- 79-0 | FEMA 3455 WS-3 | 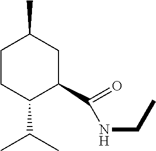 | pleasant cooling in oral cavity, ~8 min, no freezing cold or pain, or sting |
| 4681 | 68489- 09-8 | FEMA 4681 WS-12 | 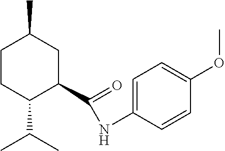 [SMILES: COc1ccc(NC(=O)[C@@H]2C[C@H](C)CC[C@H]2C(C)C)cc1] | pleasant cooling in oral cavity, ~20 min, no freezing cold or pain, or sting |
| 4309 | 68489- 14-5 | FEMA 4309 WS-5 | 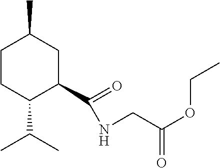 [SMILES: CCOC(=O)CNC(=O)[C@@H]1C[C@H](C)CC[C@H]1C(C)C] | pleasant cooling in oral cavity, ~12 min, no freezing cold or pain, or sting |
| — | 1401555- 39-2 | Ax-8 p-Menthane- Gly-OiPr Wei U.S. Pat. No. 8,426,463. 2013. | 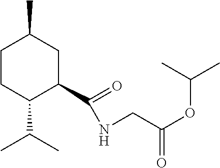 [SMILES: CC(C)OC(=O)CNC(=O)[C@@H]1C[C@H](C)CC[C@H]1C(C)C] | pleasant cooling in oral cavity, ~27 min, no freezing cold or pain, or sting |

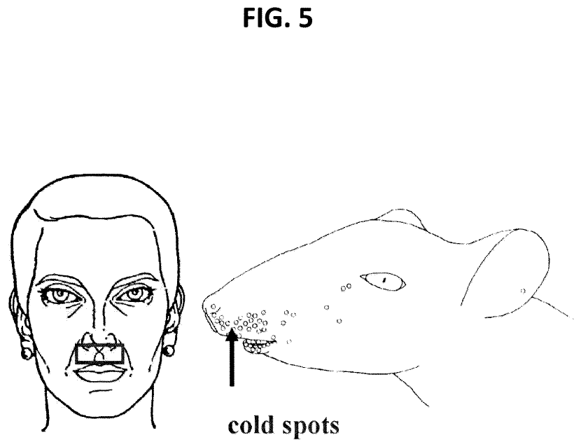

TRPM8 EC50 Receptor Assay. In 2002, groups led by David Julius and Ardem Patapoutian (Nobel Laureates of 2021) showed that menthol and icilin, both purported cooling agents, acted on a neuronal membrane protein called the TRPM8 receptor. A receptor assay is a standard tool and can be done quickly. For example, in a 384-plate assay, you can easily test 10 compounds and get dose-response data. For a chemical agonist, an in vitro TRPM8 assay generates an EC₅₀ (median effective concentration). But the quantitative correlation between the EC₅₀ on TRPM8 and cooling sensation was vague. That is, the science was “roughly” predictive, but there was considerable uncertainty for each agonist. The EC₅₀ values have scientific panache, but do not give information on the duration of action or the quality of the sensation.
Philtrum (Skin) Assay. For cooling sensations on the skin, it is necessary to test the substance on the skin. Wei proposed the use of the skin on the philtrum as a test site for cooling activity of chemicals (FIG. 5). The test substance is dissolved in a carrier, such as solvent, gel, or ointment and applied to the philtrum, a surface area of ˜10 to 12 cm². The subject records sensations on the philtrum and environs as:

The subject is given a form to record observations and instructions to mark sensations on the philtrum and environs as: 0=no changes in sensation, 1=a slight feeling of coolness, cold, or tingling, 2=a clearcut feeling of coolness or cold, and 3=a robust feeling of coolness and cold.

The intervals for measurements are usually at 5, to 15 min after application, until two successive zeros are obtained. The philtrum application site is the midline groove above the upper lip border and vermilion. The gel is placed on the philtrum with a cotton-tipped applicator up to the nasolabial folds and sometimes, but not deliberately, on the lower nostrils (subnasale). Cold receptors densely innervate the philtrum skin (FIG. 5), second only to the surfaces of the eyeball and anogenitalia. The lips also have a high density of “cold spots.” Hensel et al. mapped the cold spots on the human face (cited in Dhaka et al., 2008). In animals such as the rat the mystacial pad (nose pad with vibrissae) is abundantly labeled with TRPM8-fibers. “Cold Points” linked to single-unit neuronal discharges can be measured in experimental animals. Wei has direct experience in such experiments (Poree et al., 1989).

Dip a cotton-tipped applicator (Puritan Cotton-tipped applicators, or Q-Tips) into a reservoir bottle containing the liquid or semi-liquid (gel) test substance. The composition adhering to the cotton (about 0.1 mL) is put on the skin. The philtrum site is shown in a cartoon. A subject can practice the method in front of a mirror with a high-intensity light source. Results for test on the philtrum are in Table 8 and FIG. 5.
See data in FIG. 6. Cooling agents, prepared as an 8% wt./vol. gel, was tested topically on the philtrum skin assay. The duration of cooling was recorded (min on y-axis). The startling activity of FEMA 4557, cooling for ˜3 hr, was unexpected and surprising. FEMA 3784 is also known as Cool 10 or menthoxypropanediol.

The preferred embodiments are shown in Table 9. Note that where R=ethyl, the compound Is FEMA 4557 and has been described in Roswell, Hems and Spring, U.S. Pat. No. 4,296,255 Oct. 20, 1981. The isopropyl, n-propyl cyclopropyl, or acetyl analogs have not been synthesized and tested. There is reason to believe that these analogs may be more potent than the ethyl analog. The extra methyl group forming the “hook” or “coat hanger” on FEMA 4557 is critical for the practice of this invention. Permeation through the stratum corneum is not an essential factor for success because FEMA 4557 is much more active than WS-23.

### Formulation, Excipients, and Initiators

The formulation of skin care products is a secretive process. Success may depend on slight changes in the selection or levels of ingredients without an obvious explanation for differences. Here, the choice of the carbomer polymer for gelling is straightforward, and varying the concentration will determine the appropriate gel viscosity.

TABLE 8
Results with Test Substances, see FIG. 6 graph.

| | % | Dura- | |
|---|---|---|---|
| Chem- | Conc. | tion | |
| ical | wt/vol | (min) | Comments |
| FEMA | 8 | 180+ | icy cold sensations, prolonged cooling, |
| 4557 | | | anti-itch effects on flea bites and other skin |
| | | | irritations such as pollen allgergies, |
| | | | qualitatively and quantitatively different |
| | | | from the other test analogs, no irritation of |
| | | | the skin was observed with FEMA 4557 |
| | | | and concentrations of up to 12% |
| | | | weight/volume applied to the philtrum skin. |
| | | | The average time of cooling for 2.4 and 8% |
| | | | concentrations were 50, 95 and 175+ min, |
| | | | respectively. |

Other compounds tested on the philtrum assay at 8%.

| | | | |
|---|---|---|---|
| WS-23 | 8 | 25 ± 3 | short duration of action, no overt cold, but |
| | | | more coolness |
| WS-12 | 8 | 38 ± 4 | pleasant cool sensations on skin |
| WS-5 | 8 | 51 ± 4 | pleasant cool sensations of skin |

The dispersion of FEMA 4557, the bulk ingredient, is facilitated by ethanol and, surprisingly, by about an added 1% by weight of l-menthol. The menthol helps FEMA 4557 to be homogeneous and dispersed in the gel. Ethanol, as a solvent, Inhibits TRPM8 agonist binding, but a concentration of ethanol <1% wt/vol reduces this Interference.

TABLE 9
Structure of preferred embodiments.
Formula 1
wherein R is ethyl, isopropyl, n-propyl, cyclopropyl, or acetyl

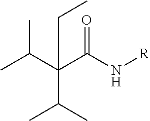

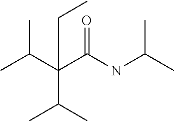

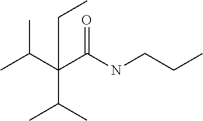

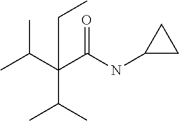

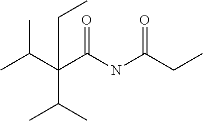 [SMILES: CCC(=O)NC(=O)C(CC)(C(C)C)C(C)C]

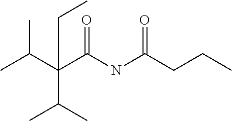 [SMILES: CCCC(=O)NC(=O)C(CC)(C(C)C)C(C)C]

FEMA 4557 is R = ethyl

Another ingredient for the formulation is an initiator of cooling. An 8% wt/vol gel of FEMA 4557 alone has an onset of 10 to 15 min for producing cool and cold. An initiator added to the formulation immediately makes the cold within 1 to 3 min and sustains the anti-itch effect. The initiator enhances the patient's appreciation of the anti-itch impact. L-Menthol works well as an initiator. Wei in U.S. Pat. No. 10,195,217 2019 describes the chemistry of DAPA compounds as skin cooling agents. The use of initiators is part of the current discovery process.

### Therapeutic Applications, Different Forms of Canine Dermatitis

The compositions and formulations contemplated here have utility in canine dermatitis conditions shown in Table 10.

Ectoparasites. A parasite is an organism that lives on or in a host and gets it food from the host. The ectoparasites that live on or in the skin of dogs belong to the arthropod phylum and include fleas, ticks, mites, and lice. For example, the mites that cause itch in dogs include Sarcoptidae and Decmodecidae. Sarcoptes scabiei cause sarcoptic mange in domestic ranging animals. The mange mite is similar to an arachnid and has four pairs of legs as an adult. By contrast, the flea is an insect with three pairs of legs. The dog flea belongs to the genus Ctenocephalides. Mites are barely visible at <1 mm, whereas the flea at ˜2 to 3 mm visibly hops around in the hair. The burrowing of the mite, its allergenic potential, and the flea bite with its saliva and secretions cause intense skin itching in the dog. Flea prevention with afoxolaner (Nexgard), a prescription drug, is a multi-billion dollar business. Like head lice in children, mites in dogs are treatable with topical scabicide solutions such as permethrin. The allergic reaction and scratching of the dog skin in response to mites and fleas can persist for weeks after removing the ectoparasites.

Fungal infestations of the skin are common in humans and other mammals. Witness the high incidence (˜10-15%) of Athlete's foot (Tinea pedis) and flaky dandruff. In dogs some common fungi and yeast are Dermatophytosis (ringworm) and Malassezia. Fungi grow well in warm and moist conditions and on the skin of dogs with thick hair. Fungi infections of the skin are irritating and cause intense scratching. Shampoos containing antifungal conazoles (ketoconazole and miconazole) kill fungi, but only if drug delivery succeeds. The 1% conazole solutions sold as OTC products are less effective than 2% prescription solutions. And the latter are expensive. Users of the shampoos need instructions to soak the infected skin for about 15 min to get adequate drug exposure and to repeat the shampoo within several days to eradicate the fungi better. However, adherence to such procedures is variable, and treatment of fungi infection is erratic. Chlorhexidine is sometimes added to anti-fungal shampoos but it has more value if there is a concurrent bacterial infection.

Atopic dermatitis (AD) in dogs is a common (5 to 15%) chronic inflammatory skin disease with altered skin barrier function and hypersensitive reactions to external allergens. No specific diagnostic test is available for AD, and the primary signs are itchiness, scratching, and damaged skin. Affected areas are frequently the belly, behind the legs, and around the base of the tail. AD may be seasonal. Susceptible breeds are retrievers (Golden and Labrador) and terriers (Wheaton and West Highland), but all species are at risk. Atopic dermatitis is relentless and not curable, and signs manifest when the dog is young. Affected dogs have a diminished quality of life, and incessant scratching and secondary infections strain the bond between dog and owner.

An effective drug for AD is oclacitinib (Apoquel, Zoetis Corp.). Oclacitinib inhibits the function of pruritogenic and pro-inflammatory cytokines dependent on JAK1 or JAK3 enzyme activity but not the hematopoietic JAK2. Oclacitinib is not a corticosteroid or an antihistamine but a specific JANUS kinase inhibitor. Oclacitinib has a fast onset of action and is effective in 50 to 66% of treated subjects. The drug requires a prescription from a veterinarian and is priced (as of 2023) online at ˜$75 per month. Adverse effects on the drug brochure include vomiting, lethargy, anorexia, diarrhea, elevated liver enzymes, dermatitis, seizures, polydipsia, and demodicosis. Nevertheless, the drug has a good profile, and JANUS kinase inhibitors are now at the forefront of pharmacology

TABLE 10
Therapeutic Indications for FEMA 4557 on Canine Skin

| Indication | Sites of Irritation | QoL |
|---|---|---|
| ectoparasites: bites | whole body scratching, increases | intense |
| from fleas and mites | risks of infection | discomfort |
| fungal and bacterial | belly, armpits, groin, “hot spots” | scratching further |
| infections, allergies | on the back and base of tail, | injures tissues |
| | suppuration and inflammation | |
| atopic and atopic- | whole body irritation | lifetime disease |
| like dermatitis | | |

The three clinical conditions have these features in common: The skin irritation and scratching is a dominant symptom that destroys the quality of life (QoL) for the dog.
The philtrum assay is ideal for finding the active ingredient with anti-irritant properties.
The preferred duration of action in the philtrum assay is >3 hr cooling, with another 3 hr residual cooling, and preferably longer.
The proposed treatment is topical and repeated. An essential goal is to reduce scratching behavior.
The formulation for delivery is easy to prepare and utilizes a “pen” for delivery (FIG. 8). A topical gel is an alternative.
The application of the drops does not cause avoidance behavior, indicating the absence of pain or discomfort.

### Comparison to JANUS Kinase Inhibitors

For canine atopic dermatitis a significant advance has been the use of oclacitinib, a specific JANUS kinase inhibitor. This drug, introduced in 2013, is an oral tablet administered once or twice a day and may have anti-scratching effects as early as four hours after dosing. Oclacitinib requires a prescription from a veterinarian and is priced at about $75 per month. It acts as an immunosuppressant and does not cure the underlying hyperactivity of the immune response. Dosing has to be maintained for the lifetime of the dog. Side-effects from oclacitinib listed on the prescribing information brochure include diarrhea, lethargy, abnormal white blood cell counts, and increase risks of infestations with mites. The use of FEMA 4557 for atopic dermatitis employs a different strategy and summarized in Table 11.

TABLE 11
Comparison of a JANUS Kinase Inhibitor and
FEMA 4557 Cooling Agent for Itch Relief.

| Parameter | -citinib (Apoquel) | FEMA 4557 Itch Relief |
|---|---|---|
| Target | JANUS-kinase | TRPM8 |
| Receptor | enzyme | ion channel on neuron |
| Mechanism | Inhibit of cytokine activation | TRPM8 agonist, cooling |
| | of inflammatory pathways | |
| Delivery to | Systemic (via bloodstream) | Topical |
| Target | | |
| Receptive | Enzymes in lymphocytes | Nerve endings in skin |
| field | | |
| Sensory | None | Cooling |
| Effect | | |
| Formulation | Oral tablet, once a day | Liquid or Gel, apply b.i.d., |
| | | t.i.d. p.r.n., ~3 |
| | | times per day |
| Route of | Oral, GI absorption | Topical and Localized, |
| delivery | | site of itch |
| Onset | 15 min, hours | ~10 min |
| Side-effects | Complex, see Prescribing | None observed |
| | Insert for Apoquel | |
| Clinical | Atopic Dermatitis | Any itch, suppression |
| Indication | | of itch-scratch cycle |
| Mol. Wt. | 337 | 199 |
| Status | Rx, requires Vet prescription | OTC |
| Costs | ~$75 per month, lifetime cost | $ less expensive |

In humans specific inhibitors of JANUS kinase are approved for AD, psoriasis and rheumatoid arthritis, but use of these drugs are carefully monitored because of potential serious side-effects such as cardiovascular risks. These drugs will not be useful for itch for insect bites, contact dermatitis, or other skin allergies. The use of a FEMA 4557 would be the preferred first choice in humans and dogs because cooling has a broad spectrum of actions (FIG. 1) and topical use has a good safety profile.

### Case Study Examples

### Study 1

### Dose-Response Study on Philtrum (see FIG. 7)

Canine skin is thinner than human skin by about 50%. The stratum corneum has about 47 cell layers and a thickness of 13 μm. The living epidermis has 3 to 6 cell layers and a thickness of 10 μm. In dogs, the hair may grow from compound hair follicles. These follicles are surrounded by 3 to 15 secondary hairs growing out of the same pore. Sebaceous glands within the skin lubricate the hair, keeping the coat shiny and water resistant. The thickness of the hair coat varies with the breed. The hair coat hinders the delivery of a topical drug to its receptors in the basal layer of the epidermis.

The test solutions were prepared using FEMA 4557 dissolved in a solvent containing racemic 1,2-propanediol, 4% ethanol, and 4% l-menthol. All percentages are given as weight/volume, so an 8% solution, for example, contains 8 g of FEMA 4557 IN 100 g of solvent. The solutions prepared were 0%, 4%, 8%, 16%, 32%, and 64%. The solution was applied to the philtrum with a cotton-tipped applicator. The volume off-loaded per application, obtained by weighing the solution before and after use, ranged from 55 μl to 85 μl, averaging 60 μl. For a 16% solution, the dose is equivalent to 160 mg/ml×60 μl=9.6 mg or about 10 mg. This delivered dose to a philtrum surface of 1 to 2 cm² is 5 to 10 mg/cm². The recording of the intensity of sensations from the philtrum is as noted above. The results of FIG. 7 lead to these conclusions.

The solubility of FEMA 4557 (≥64%) facilitate delivery to receptors. As shown in FIG. X, FEMA 4557 is miscible with the solvent and forms a transparent solution at room temperature. This property enhances the delivery of the active agent to the receptive field, located in the epidermal layers of the organism's skin below the stratum corneum. For the canine skin, the barriers to drug delivery are the dense hair follicles, which may number several hundred thousand per cm². Hair density for the dog, on average, is 15× greater than hair on the human scalp. A liquid will wick down the hair shaft via surface tension and distribute better than any solid delivery vehicle, such as a gel or cream, which will adhere to the surface of the hair shaft. A liquid is quickly delivered and distributes the active ingredient evenly onto the skin's surface. This delivery mechanism is impossible with other compounds such as WS-5, WS-12, or WS-30 because they are not as soluble as FEMA 4557.

Absence of adverse sensations of stinging cold. The tested FEMA 4557 solution at ≥64% on the philtrum produced a pleasant and robust cool. No cold stinging sensations were felt. This is contrasted to DIPA-1-7 (Cryosim-1), another agent with good skin penetrability but which produces a frigid cold and irritation. This safety feature of FEMA 4557 was unexpected and beneficial.

Sufficient duration of action. As can be seen from the graph, a single dose of the 16 to 32% will provide ˜3 hr of cooling. This effect is accompanied by an “after effect” of another 3 hr cooling on the skin. A warm towel applied to the philtrum skin will reveal this aftereffect. That is, there is a memory, reservoir, or residual cooling that linger and supplement the drug action on dysesthesia. The total therapeutic duration is at least 6 hr at 16 to 32% and provides sufficient daily control of dysesthesia at a b.i.d., t.i.d, or p.r.n. dosing. Other test agents did not have a similar duration of action.

Practice of invention. The potency of FEMA 4557, costs of production of the active ingredient, solubility in biocompatible solvents, ease of formulation into a liquid for delivery, absence of side effects, cooling efficacy, and duration of therapeutic action are unique and surprising. No other chemical tested by the applicant has all of these desirable properties.

### Study 2

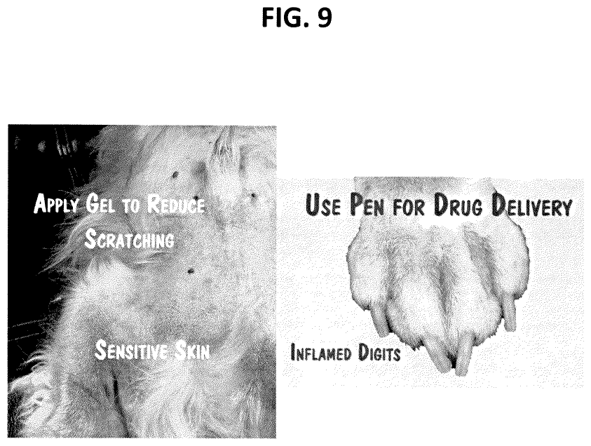

A six-year-old long-haired Dachshund, over eight months, developed an inflamed area on the belly and lower right groin where it frequently licked and scratched. Upon examination, the site was red and swollen (FIG. 9). A visit to the veterinarian ruled out fleas and mites or fungal and bacteria growth. The veterinarian called it a non-specific allergy and recommended cetirizine hydrochloride liquid compounded for dogs. Taking cetirizine (Zyrtec, an antihistamine) for one week did not affect the red skin or the licking. The Applicant gave the dog's owner an airless pump dispenser containing 8% wt/vol of FEMA 4557 dispersed in 4 mg/ml of carbapol 980, formulated with 1% ethanol and 1% menthol, and instructed him to apply the gel to the skin of the dog's belly and groin. Within one day after the application of FEMA 4557, scratching and licking were reduced, and after three days of daily application, the redness and swelling disappeared. Treatment continued for one week, after which the skin appeared utterly normal. The inflammation and scratching have not come back.

### Study 3

A twelve-year-old West Highland Terrier had periodic episodes of seasonal skin allergy. She would scratch until the site (groin and hindquarters) lost its fur and was bare down to the skin. A visit to the vet concluded that fleas, mites, or pyoderma were not causing the skin irritation, but the condition may be due to fungus. The vet recommended an antifungal shampoo, but repeated bathing with a ketoconazole or miconazole shampoo did not make any difference. The owner of the dog, who had access to a pharmacy, applied nystatin ointment and a potent skin steroid cream containing triamcinolone acetate. The treated dog initially seemed to respond but then licked its inflamed skin and occasionally “chased its tail” as it tried to reduce discomfort by biting. However, the skin remained inflamed, looked fragile, and oozed liquid, and the paws became red from excessive licking.

An 8% FEMA 4557 gel spread on the bare spots of the groin, hindquarters, and perianal area stopped the scratching. Next, a 20% wt/vol FEMA 4557 solution, dissolved in propylene glycol, was applied to the interdigital folds of the paws with a cotton-tipped applicator (FIG. 9). The dog stopped licking its front paws. All signs of skin discomfort disappeared after five days of treatment with FEMA 4557, and the bare spots began to grow hair.

In summary, a chemical entity, FEMA 4557, was identified as a chemical entity that a) has a rapid onset of action, b) can be produced, formulated, and delivered at reasonable costs, c) is free of adverse side effects, and d) reduce dysesthesia, inhibits scratching, and progression of the itch-scratch cycle. The treatment of canine skin disorders with this molecule should accelerate skin healing.

### REFERENCES

A number of publications are cited herein in order to describe and disclose the invention and the state of the art to which the invention pertains. Citations for these publications are alphabetized and provided below for easy reference.
Behrendt, H-J. et al., Characterization of the mouse cold-menthol receptor TRPM8 and vanilloid receptor type-1 VR1 using a fluorometric imaging plate reader (FLIPR) assay. Brit. J. Pharmacol. (2004) 1-9.
Bíró T, Ko M C, Bromm B, Wei E T. et al. How best to fight that nasty itch—From new insights into the neuroimmunological, neuroendocrine, and neurophysiological bases of pruritus to novel therapeutic approaches. Exp Dermatol. 2005; 14(3).
Bödding M, et al. Characterisation of TRPM8 as a pharmacophore receptor. Cell Calcium. 2007; 42(6):618-28.
Cevikbas F, Lerner E A. Physiology and pathophysiology of itch. Physiol Rev. 2020; 100(3):945-82.
Dhaka et al., 2008. TRPM8-expressing sensory neurons and their projections. J Neurosci. 2008 Jan. 16: 28(3):566-75.
Erman, M. B. Cooling Agents and Skin Care Applications. Cosmetics and Toiletries Magazine, Vol. 120, pg. 105-118, 2005.
Feldberg W. John Henry Gaddum. 1900-1965. Memoirs of the Fellows of the Royal Society. 1967; (November):56-77).
Johnson S et al. Trigeminal Receptor Study of High-Intensity Cooling Agents. J Agric Food Chem. 2018; 66(10):2319-23.
Jung M J, Wei E T. et al. A randomized, vehicle-controlled clinical trial of a synthetic TRPM8 agonist (DIPA-1-7) gel for itch. J Am Acad Dermatol. 2021; 84(3):869-71.
Kang S Y, Wei E T, et al. TRPM8 agonist (cryosim-1) gel for scalp itch: A randomized, vehicle controlled clinical trial. J Eur Acad Dermatology Venereol. 2022; 1-2.
Leffingwell J, Rowsell D. Wilkinson Sword cooling compounds: from the beginning to now. Perfume & Flavorist 39: 34-44, 2014.
Leffingwell, J. C. Cooling ingredients and their mechanism of action. Handbook of cosmetic science and technology. Chpt. 65. Pp. 661-675. Barel, Paye, and Maibach. (edited). Informa Health Care, New York, 2009.
McKemy D D, Neuhausser W M, Julius D. Identification of a cold receptor reveals a general role for TRP channels in thermosensation. Nature. 2002; 416(6876):52-8.
Raef H S, Elmariah S B. Vulvar Pruritus: A Review of Clinical Associations, Pathophysiology and Therapeutic Management. Front Med. 2021; 8(April):1-10.
Poree L R, Dickenson A H, Wei E T. Corticotropin-releasing factor inhibits the response of trigeminal neurons to noxious heat. Brain Res. 1989 Nov. 20: 502(2):349-55.
Roswell D G et al. Acyclic carboxamides having a physiological cooling effect. U.S. Pat. No. 4,296,255. Oct. 20, 1981.
Sherkheli M A et al. Characterization of Selective TRPM8 Ligands and their Structure. 2010; 13(2):242-53.
Sherkheli M A, et al., Characterization of Selective TRPM8 Ligands and their Structure. 2010; 13(2):242-53.
Smith R L et al. GRAS flavoring substances 24. Food Technol. 2009; 63(6):46-62.
Steinhoff M, E T Wei, Neurophysiological, neuroimmunological, and neuroendocrine basis of pruritus. J Invest Dermatol. 2006 August; 126(8):1705-18.
Sun, H S. Compound with physiological cooling effect. U.S. Pat. No. 7,030,273. Apr. 18, 2006.
Wei, E T. 1,2,3,6-Tetrahydropyrimidine-2-one compositions and therapeutic methods therewith for pain and inflammation. U.S. Pat. No. 6,743,801. Jun. 1, 2004.
Wei, E T. [((1R,2S,5R)-2-Isopropyl-5-methyl-cyclohexanecarbonyl)-amino]-acetic acid isopropyl ester and related compounds and their use in therapy. U.S. Pat. No. 8,426,463. Apr. 23, 2013.
Wei, E T. N-Alkylcarbonyl-amino acid ester compounds and their use for skin irritation, itch and pain. U.S. Pat. No. 6,599,524. Oct. 7, 2014.
Wu Z H et al. Acute and subacute inhalation toxicity assessment of WS-23 in Sprague-Dawley rats. J Appl Toxicol. 2021; 41(11):1826-38.
Xu et al. (Molecular mechanisms underlying menthol binding and activation of TRPM8 ion channel. Nat Commun.] 2020. http://dx.doi.org/10.1038/s41467-020-17582-x)
Yogeswaran S et al. The role of Synthetic Coolants, WS-3 and WS-23, in modulating E-cigarette-induced Reactive Oxygen Species (ROS) in lung epithelial cells. Toxicol Reports [Internet]. 2022; 9(June):1700-9. https://doi.org/10.1016/i.toxrep.2022.08.007

1. A method for the treatment of a dermatological disorder in a hair-coated mammal in need of treatment thereof, comprising: topically applying a therapeutically effective amount of a liquid or semi-liquid composition of N-ethyl-2,2-diisopropyl butanamide.

2. The method as in claim 1 wherein the composition has from about 5% to 65% by weight of N-ethyl-2,2-diisopropyl butanamide.

3. The method as in claim 1 wherein the amount of N-ethyl-2,2-diisopropyl butanamide in the composition is from about 2 to 20 mg per dose.

4. The method as in claim 1 wherein the hair-coated mammal in need of treatment is the domesticated dog (Canis familiaris).

5. The method as in claim 1 wherein the dermatological disorder in need of treatment is the excessive scratching caused by dermatitis.

6. The method as in claim 1 wherein the dermatological disorder in need of treatment is the inflammation of canine dermatitis.

7. The method as in claim 1 wherein the dermatological disorder in need of treatment is the scratching caused by the bites of insects.

8. The method as in claim 1 wherein the dermatological disorder in need of treatment is the scratching caused by the bites of ectoparasites.

9. The method as in claim 1 wherein the dermatological disorder in need of treatment is the scratching caused by fungal, yeast, or bacterial infections of the skin.

10. The method as in claim 1 wherein the dermatological disorder in need of treatment is the scratching caused by allergy.

11. The method as in claim 1 wherein the irritation of the dermatological disorder in need of treatment is the inflammation of canine atopic dermatitis or canine atopic-like dermatitis.

12. The method as in claim 1 wherein the dermatological disorder in need of treatment is the excessive scratching behavior caused by canine atopic dermatitis or canine atopic-like dermatitis.

13. The method as in claim 1 wherein the dermatological disorder in need of treatment is the itch-scratch cycle of dermatitis.

14. The method as in claim 1 wherein the irritation of the dermatological disorder in need of treatment is the inflammation caused by folliculitis (pyoderma traumatic), acne, or malaria rubra (heat rash).

15. The method as in claim 1 wherein the dermatological disorder being treated in the hair-coated mammal includes those sensations that would be interpreted by humans as burning sensations, itch, and pain.

16. The method as in claim 15 wherein the dermatological disorder in need of treatment is the itch and irritation of inflamed skin.

17. A pharmacological composition for the treatment of a dermatological disorder in a hair-coated mammal in need of treatment thereof, comprising:
a liquid or semi-liquid composition having a therapeutically effective amount of one or more compounds having Formula 1 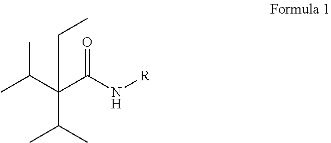
wherein R is ethyl, n-propyl, isopropyl, cyclopropyl, or acetyl

18. A therapeutic method for the treatment of the irritation of a dermatological disorder in a mammal in need of treatment thereof, comprising:
topically applying a liquid or semi-liquid composition to the hair-coated mammal's skin, the composition comprising a therapeutically effective amount of one or more compounds having Formula 1 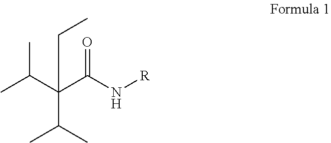
wherein R is ethyl, isopropyl, n-propyl,r cyclopropyl, or acetyl and a second cooling agent comprising l-menthol.

19. The method as in claim 18 wherein the composition has from about 2 to 5% by weight of l-menthol.
